(19) United States
(12) Patent Application Publication
(10) Pub. No.: US 2002/0197694 A1
(43) Pub. Date: Dec. 26, 2002

(54) Conjugates of reduced antibodies and biomolecules

(76) Inventor: Weiping Shao, Cheshire, CT (US)

Correspondence Address: NEEDLE & ROSENBERG, P.C., The Candler Building, 127 Peachtree Street, N.E., Atlanta, GA, 30303-1811, US

(21) Appl. No.: 10/143,517

(22) Filed: May 9, 2002

Related U.S. Application Data
(60) Provisional application No. 60/299,671, filed on Jun. 20, 2001.

(51) Int. Cl. A61K 39/395; C12N 9/00; C07K 16/46

(52) U.S. Cl. 435/188.5; 530/391.1; 424/178.1

(57) ABSTRACT

Disclosed are compositions containing antibody conjugates made up of an antibody fragment and a biomolecule. The biomolecule is coupled to the antibody fragment via a reactive chemical group such that the coupling between the biomolecule and the antibody fragment is resistant to reducing agents. Reactive chemical groups include sulfhydryl groups, amino groups, carboxyl groups, and imidazole groups. The reactive chemical group can be in the hinge region of the antibody fragment. This location reduces or eliminates interference between the antibody/antigen interaction and the biomolecule. The biomolecule can be coupled to the antibody fragment via a maleimide group. The antibody fragment preferably is a half antibody or a F(ab′)₂. Half antibodies can be produced by reducing an antibody to break disulfide bonds.

### CROSS-REFERENCE TO RELATED APPLICATIONS

This application claims benefit of U.S. Provisional Application No. 60/299,671, filed Jun. 20, 2001, which application is hereby incorporated herein by reference in its entirety.

### FIELD OF THE INVENTION

The disclosed invention is generally in the field of antibody conjugates and analyte detection methods.

### BACKGROUND OF THE INVENTION

The antigen-antibody interaction is a bimolecular association similar to an enzyme-substrate interaction, with the important distinction that it is a reversible process. The interactions between an antibody and antigen are governed by various noncovalent interactions between the antigenic determinant, or epitope, of the antigen and the variable-region domain of the antibody molecule. The specificity of an antibody for an antigen has led to the development of a variety of immunologic assays which can be used to detect the presence of antibody or antigen. These assays have been instrumental in diagnosing diseases, monitoring the level of the humoral immune response, and identifying molecules of biological interest.

Antigens are routinely detected on membranes (Western blots) and in situ (immunohistochemistry, immunofluorescence, immunostaining, etc.) There are many variations on the available methods of detecting antigens, depending on the number and types of antibodies used, the label and the substrate. Independent of the variation, antigen detection essentially depends upon a specific antibody-antigen reaction forming an antibody-antigen complex.

The noncovalent interactions that comprise antigen-antibody binding include hydrogen bonds, and ionic, hydrophobic and van der Waals interactions, each of which is relatively weak in comparison to a covalent bond, and with each effective interaction operating over a very small distance. Therefore, a strong antigen-antibody interaction requires a large number of such associations, and a very tight fit between the antigen and antibody, owing to the high degree of specificity which is characteristic of antigen-antibody interactions.

The detection of the primary antibody-antigen complex has been demonstrated in numerous ways. Detection methods include directly labeled monoclonal antibody, wherein the label consists of an enzyme, e.g., alkaline phosphatase (AP), and Horseradish Peroxidase (HRP); a fluorochrome (a fluorescent compound), e.g., fluorescein, rhodamine, Texas Red, Cy-3, and Cy-5; a heavy metal chelate such as europium, lanthanum, yttrium, and gold; a radioactive isotope; or the label may be a secondary reporter, e.g., biotin, streptavidin, avidin, digoxigenin, or dinitrophenyl. Alternatively, detection methods may also include directly labeled polyclonal antibody, wherein the label may consist of the above-identified elements listed for monoclonal antibodies. Further, labeled secondary antibody which is polyclonal anti-first antibody, such as goat anti-mouse IgG-conjugate, may be used as a method of detection. Other detection methods include the use of labeled secondary reagent which is not necessarily an antibody, such as AP-streptavidin; labeled secondary antibody which is anti-conjugated epitope, such as HRP-goat-antifluorescein and AP-rabbit-anti-DNP; and unlabeled secondary antibody, detected with a labeled tertiary antibody or labeled tertiary component.

In extracts where the antigenic proteins represent only a tiny fraction of the total protein, the number and sizes of proteins with a particular epitope can be rapidly determined by Western blotting. Western blotting consists of electrophoretic transfer of an antigenic protein or proteins from a sodium dodecyl sulfate-polyacrylamide gel (SDS-PAGE) onto a nitrocellulose filter placed on one face of the gel, and as the protein is transferred, its position on the SDS-PAGE gel is preserved. The transferred protein binds tightly and non-covalently to the nitrocellulose, and can be exposed to a primary antibody that will bind to it. This bound primary antibody can then be bound by a secondary antibody containing a visualizable, covalently attached marker. If labeled specific antibody is not available, antigenantibody complexes can be detected by adding a secondary anti-epitope antibody that is either radiolabeled or enzyme-labeled, and the band is visualized by autoradiography or substrate addition. Only those proteins with the epitope will be visualized in this manner, and if several proteins with different molecular weights have the epitope, each will be seen as a separate band on the nitrocellulose (S. Hockfield, et al., Selected Methods for Antibody and Nucleic Acid Probes, Cold Spring Harbor Laboratory Press, 1993, pp. 293-316).

Western blotting can identify either a given protein antigen or specific antibody. For example, Western blotting has been used to identify the envelope and core proteins of HIV and the antibodies to these components in the serum of HIV-infected individuals.

Immuno-PCR, a hybrid of PCR and immunoassay systems, combines the versatile molecular recognition of antibodies with the amplification potential of DNA replication. The technique involves the in situ assembly of the labeled DNA-antibody complex during the assay, creating variable stoichiometry in both the attachment of the DNA label, and the assembly of the components.

The procedural complexity of immuno-PCR has been reduced by the direct chemical attachment of DNA to analyte antibodies, whereby immobilized capture antibodies and a reporter antibody that carries a covalently attached DNA label are used, and the assay response is obtained by PCR of the DNA label and detection of the amplification products. This technique has been modified to develop an immuno-PCR sandwich assay for multiple analytes (see R. D. Joerger, et al., Clinical Chemistry, 1995, 41 (9): 1371-1377; E. R. Hendrickson, et al., Nucl. Acids Res., 1995, 23 (3): 522-529; and T. Sano, et al., Science, 1992, 258: 120-122).

However, immuno-PCR, albeit exhibiting enhanced sensitivity over traditional methods, is time consuming, complex and it does not lend itself to automation.

Antibody fragments of small size are of particular advantage in many applications. In diagnostic applications (e.g. ELISA, RIA, etc.), the smaller molecule's surface decreases the problems of nonspecific interactions, which are known to frequently involve the constant domains. The same is true in using antibody fragments as ligands in affinity chromatography. In tumor diagnostics or therapy, it is important that a significant proportion of the injected antibody penetrates tissues and localizes to the tumor, and is dependent on the molecular dimensions (Colcher et al., 1990, J. Natl. Cancer Inst. 82, 1191-1197). Expression yields and secretion efficiency of recombinant proteins are also a function of chain size (Skerra & Pluckthun, 1991, Protein Eng. 4, 971) and smaller proteins are preferred for this reason. Therefore, molecules of a small size are advantageous for several reasons.

### BRIEF SUMMARY OF THE INVENTION

Disclosed are compositions containing antibody conjugates made up of an antibody fragment and a biomolecule. The biomolecule is coupled to the antibody fragment via a reactive chemical group such that the coupling between the biomolecule and the antibody fragment is resistant to reducing agents. Reactive chemical groups include sulfhydryl groups, amino groups, carboxyl groups, and imidazole groups. The reactive chemical group can be in the hinge region of the antibody fragment. This location reduces or eliminates interference between the antibody/antigen interaction and the biomolecule.

The biomolecule can be coupled to the antibody fragment via a maleimide group. The antibody fragment preferably is a half antibody or a F(ab′)₂. Half antibodies can be produced by reducing an antibody to break disulfide bonds. The biomolecule can be, for example, a nucleic acid, a protein, a carbohydrate, an oligonucleotide, an oligopeptide, an oligosaccharide, a peptide, a hapten, or an aptamer. The biomolecule preferably is a nucleic acid. The disclosed antibody conjugates can also include one or more additional biomolecules. The additional biomolecules can be coupled in the same manner as the first biomolecule. The biomolecules in an antibody conjugate can be the same or different. More specifically, the biomolecules can have the same structure or different structures. For example, in the case of nucleic acids (as the biomolecules), the nucleic acids can have the same nucleotide sequence or different nucleotide sequences.

The antibody fragments in the disclosed antibody conjugates generally are specific for an antigen or analyte. Such analytes can include proteins or peptides, preferably proteins or peptides associated with a disease or condition. The biomolecule preferably is an oligonucleotide. The disclosed antibody conjugates can be coupled, linked, attached or otherwise associated with a solid support. Such compositions are useful for example, for analytical and diagnostic uses of the antibody conjugates.

A preferred way to detect an antibody conjugate is by rolling circle amplification mediated by an oligonucleotide in the conjugate. The oligonucleotide can mediate rolling circle amplification by, for example, serving as a primer for rolling circle replication, serving as a template for rolling circle replication, or serving as a target sequence in ligation-mediated rolling circle amplification.

The disclosed antibody conjugates can be used for any purpose for which antibodies can be used. Numerous such methods are known. For example, antibodies find extensive uses in analytic methods, including methods for detecting and quantitating, or involving detection or quantitation, of antigens and analytes. Antibodies also find use in diagnostic and therapeutic methods. In general, the disclosed antibody conjugates can be used to detect analytes by bringing into contact a antibody conjugate and a sample under conditions that allow interaction of the antibody conjugate and an analyte, where the antibody fragment is specific for the analyte.

### BRIEF DESCRIPTION OF THE DRAWINGS

FIG. 1 is a diagram illustrating coupling of two oligonucleotides to a reduced antibody (half antibody).

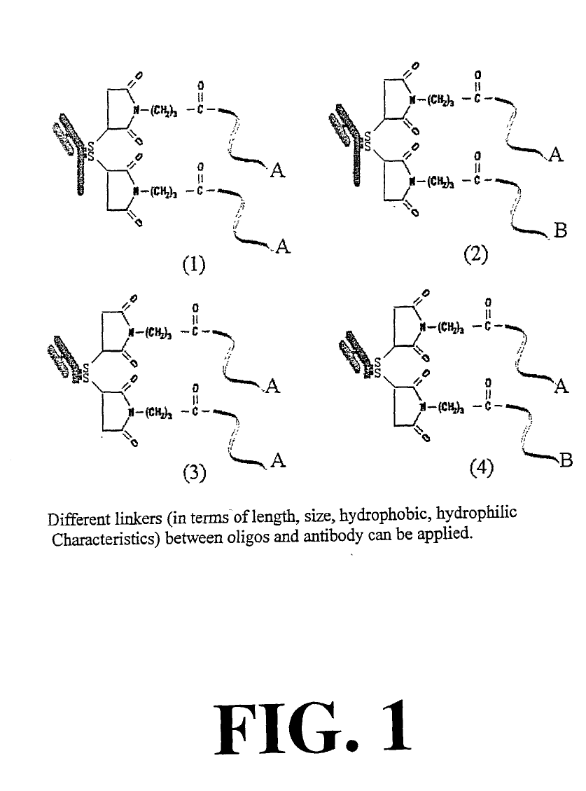

FIG. 2 is a diagram illustrating antibody conjugates involving two of the same oligonucleotides (left side) or two different oligonucleotides (right side). Conjugates involving half antibodies are shown at the top. Conjugates involving F(ab′)₂ are shown at the bottom.

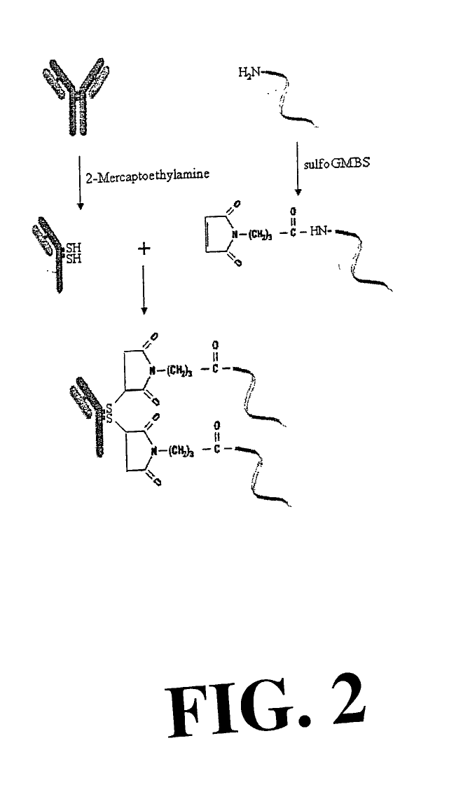

FIG. 3 is a bar graph showing the increase in detection signal intensity that is obtained using half antibody conjugates versus a whole (intact) antibody conjugate.

### DETAILED DESCRIPTION OF THE INVENTION

Disclosed are compositions containing antibody conjugates made up of an antibody fragment and a biomolecule. The biomolecule is coupled to the antibody fragment via a reactive chemical group such that the coupling between the biomolecule and the antibody fragment is resistant to reducing agents. Preferred reactive chemical groups are sulfhydryl groups, amino groups, carboxyl groups, and imidazole groups. Sulfhydryl groups are most preferred. The sulfhydryl group can be on a cysteine residue in the antibody fragment. It is preferred that the reactive chemical group be in the hinge region of the antibody fragment. This location reduces or eliminates any interference between the antibody/antigen interaction and the biomolecule. Resistance of the coupling to reducing agents makes the disclosed antibody conjugates more stable. Sulfhydryl groups can also be chemically introduced into the antibody using a thiolating agent such as 2-IT.

Preferably, the biomolecule is coupled to the antibody fragment via a maleimide group coupled to a sulfhydryl group in the hinge region of the antibody fragment. For this purpose, antibody conjugates can be made by reacting a maleimide-derivatized form of the biomolecule with the antibody fragment. More specifically, antibody conjugates can be made by reducing an antibody to produce the antibody fragment, producing an amine biomolecule, derivatizing the amine biomolecule with maleimide to produce a maleimide-derivatized biomolecule, and reacting the maleimide-derivatized biomolecule with the antibody fragment (Example 1).

A most preferred approach is to derivatize a whole antibody with thiol groups using chemicals like Traut's reagent, then reduce the antibody to produce an antibody fragment, produce an amine biomolecule, derivative the amine biomolecule with maleimide to produce a maleimide-derivatized biomolecule, and react the maleimide-derivatized biomolecule with the antibody fragment (Example 2).

In another preferred approach, amine groups on the intact antibody can be derivatized with maleimide to produce a maileimide-derivatized antibody, and then the maleimide-derivatized antibody can be reacted with a thiol-containing biomolecule. The antibody portion of this conjugate can then be reduced to produce the antibody fragment. Additional biomolecules can be coupled to this antibody fragment via a maleimide group coupled to a sulfhydryl group in the hinge region of the antibody fragment. For this purpose, antibody conjugates can be made by reacting a maleimide-derivatized form of the biomolecule with the antibody fragment. More specifically, antibody conjugates can be made by reducing an antibody to produce the antibody fragment, producing an amine biomolecule, derivatizing the amine biomolecule with maleimide to produce a maleimide-derivatized biomolecule, and reacting the maleimide-derivatized biomolecule with the antibody fragment.

The antibody fragment preferably is a half antibody or a F(ab′)₂. Such antibody fragments can still interact with antigen but exposes better sites for attachment of biomolecules (such as the hinge region of the antibody), are smaller that full antibodies (thus providing increased movement into tissue). Half antibodies can be produced by reducing an antibody to break disulfide bonds. Reduced antibodies can have higher sensitivity than intact antibodies due to more effective antigen binding.

The biomolecule preferably is a nucleic acid, a protein, a carbohydrate, an oligonucleotide, an oligopeptide, an oligosaccharide, a peptide, a hapten, or an aptamer. The biomolecule most preferably is a nucleic acid. The disclosed antibody conjugates can also include one or more additional biomolecules. The additional biomolecules can be coupled in the same manner as the first biomolecule. The biomolecules in an antibody conjugate can be the same or different. More specifically, the biomoleucles can have the same structure or different structures. For example, in the case of nucleic acids (as the biomolecules), the nucleic acids can have the same nucleotide sequence or different nucleotide sequences.

The antibody fragments in the disclosed antibody conjugates preferably are specific for an antigen or analyte. Such analytes can include haptens, drugs, or proteins or peptides, preferably proteins or peptides associated with a disease or condition. The disclosed antibody conjugates can be used in sets. In such sets, the antibody fragments, the biomolecules, or both, can be the same or different for all or some of the members of the set. In preferred sets, each antibody fragment of each antibody conjugate is specific for a different analyte.

The biomolecule preferably is an oligonucleotide. Such oligonucleotides can include detection portions, that is, a region of the oligonucleotide useful for detecting the antibody conjugate. Where multiple oligonucleotides are coupled to an antibody fragment, the detection portions of the oligonucleotides can have the same or different nucleotide sequences. The oligonucleotide can mediate detection in many ways. For example, the oligonucleotide can be detected via hybridization of a labeled probe.

A preferred way to detect an antibody conjugate is by rolling circle amplification mediated by an oligonucleotide in the conjugate. The oligonucleotide can mediate rolling circle amplification by, for example, serving as a primer for rolling circle replication, serving as a template for rolling circle replication, or serving as a target sequence in ligation-mediated rolling circle amplification. In the case of a primer, the oligonucleotide would be a rolling circle replication primer. In the case of template, the oligonucleotide would be an amplification target circle. In ligation-mediated rolling circle amplification, the target sequence serves as a hybridization partner that brings that ends of a linear nucleic acid molecule into proximity with each other such that ligation of the ends results in circularization of the linear molecule. The circularized nucleic acid molecule can then serve as a template for rolling circle amplification.

The disclosed antibody conjugates can be coupled, linked, attached or otherwise associated with a solid support. Such compositions are useful for example, for analytical and diagnostic uses of the antibody conjugates. The solid support can be in any form. Examples include thin film, membrane, bottles, dishes, slides, fibers, woven fibers, optical fibers, shaped polymers, particles, beads, microparticles, or a combination. The solid support can be made of any material. For example, the solid support can be made of acrylamide, agarose, latex, cellulose, nitrocellulose, glass, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, polyamino acids, or a combination. The solid support can be porous or non-porous.

Preferred forms of the disclosed antibody conjugate/solid support compositions have a plurality of antibody conjugates. In such a set of antibody conjugates, the antibody fragments, the biomolecules, or both, can be the same or different for all or some of the members of the set. In preferred sets, each antibody fragment of each antibody conjugate is specific for a different analyte. The antibody conjugates can be located in the same or different regions of the solid support. Preferably, the antibody conjugates are located in predefined regions of the solid support. The distance between the different predefined regions of the solid support can be fixed, variable or a combination. For example, the distance between at least two of the different predefined regions of the solid support can be variable.

The disclosed antibody conjugates can be used for any purpose for which antibodies can be used. Numerous such methods are known. For example, antibodies find extensive uses in analytic methods, including methods for detecting and quantitating, or involving detection or quantitation, of antigens and analytes. Antibodies also find use in diagnostic and therapeutic methods. In general, the disclosed antibody conjugates can be used to detect analytes by bringing into contact a antibody conjugate and a sample under conditions that allow interaction of the antibody conjugate and an analyte, where the antibody fragment is specific for the analyte.

### Materials

Antibody Conjugates

Antibody conjugates are antibody fragments to which one or more biomolecules have been attached. The coupling between the antibody fragment and a biomolecule is resistant to reducing reagents. The biomolecule is coupled to the antibody fragment via a reactive chemical group on the antibody fragment. Antibody conjugates can have a single biomolecule or multiple biomolecules. Preferred forms of antibody conjugates have two biomolecules attached via two different reactive groups. Where multiple biomolecules are attached to an antibody fragment, the biomolecules can be the same or different, can have the same structure or different structures.

Multiple biomolecules of the same structure are useful for a variety of purposes including increasing the reactivity, effectiveness, or detectability of the biomolecule. Use of multiple biomolecules having different structures allows combinations of effects with the same antibody conjugate. Where the biomolecules in an antibody conjugate are used for detection, mutliple different biomolecules allows the biomolecule “signal” from the antibody conjugate to be encoded. The different combinations of biomolecules on the antibody conjugates identifies the specific conjugate.

As used herein, coupled and coupling refer to linkage or attachment of two components via one or more covalent bonds. A coupling that is resistant to reducing agents refers to resistance of bonds in the linkage of two coupled components to breakage by a reducing agent. Resistance does not refer only to complete stability (that is, the absence of bond breakage), but includes a reduction in bond breakage compared to non-resistant linkages such as disulfide bonds.

Antibody Fragments

Antibody fragments are portions of a complete antibody. A complete antibody refers to an antibody having two complete light chains and two complete heavy chains. An antibody fragment lacks all or a portion of one or more of the chains. Preferred antibody fragments are half antibodies and fragments of half antibodies. A half antibody is composed of a single light chain and a single heavy chain. Half antibodies and half antibody fragments can be produced by reducing an antibody or antibody fragment having two light chains and two heavy chains. Such antibody fragments are referred to as reduced antibodies. Reduced antibodies have exposed and reactive sulfhydryl groups. These sulfhydryl groups can be used as reactive chemical groups or coupling of biomolecules to the antibody fragment. A preferred half antibody fragment is a F(ab). The hinge region of an antibody or antibody fragment is the region where the light chain ends and the heavy chain goes on.

Antibody fragments for use in antibody conjugates can bind antigens. Preferably, the antibody fragment is specific for an antigen. An antibody or antibody fragment is specific for an antigen if it binds with significantly greater affinity to one epitope than to other epitopes. The antigen can be any molecule, compound, composition, or portion thereof to which an antibody fragment can bind. An analyte can be any molecule, compound or composition of interest. Preferred antigens and analytes are proteins and peptides. The protein or peptide can be a protein or peptide associated with a disease or condition.

Antibody fragments can be used to bind analytes. In general, any compound, moiety, or component of a compound or complex can be an analyte. Preferred analytes are peptides, proteins, and other macromolecules such as lipids, complex carbohydrates, proteolipids, membrane fragments, and nucleic acids. Analytes can also be smaller molecules such as cofactors, metabolites, drugs, haptens (e.g. biotin), enzyme substrates, metal ions, and metal chelates. Analytes preferably range in size from 100 daltons to 1,000,000 daltons.

Analytes may contain modifications, both naturally occurring or induced in vitro or in vivo. Induced modifications include adduct formation such as hapten attachment, multimerization, complex formation by interaction with other chemical moieties, digestion or cleavage (by, for example, protease), and metal ion attachment or removal. The disclosed method can be used to detect differences m the modification state of an analyte, such as the phosphorylation or glycosylation state of proteins.

Analytes can be associated directly or indirectly with substrates, preferably in arrays. Most preferred are microarrays. Analytes can be captured and/or immobilized using the disclosed antibody conjugates. Alternatively, immobilized analytes can be used to capture the disclosed antibody conjugates.

Biomolecules

Biomolecules are molecules that are present in cells, are a type of molecule found in cells, or that have a biological effect. Examples include nucleic acids, proteins, carbohydrates, oligonucleotides, oligopeptides, oligosaccharides, peptides, haptens, aptamers, drugs, and toxins. The type of biomolecule and the specific form of biomolecule used will generally depend on the intended use for the antibody conjugate. For example, a drug or toxin for therapeutic use, or a nucleic acid or protein for detection.

Preferred biomolecules are nucleic acids, such as oligonucleotides. Oligonucleotides can consist of unmodified or modified nucleotides or other functional groups or a mixture of these components. Oligonucleotides are particularly suited for use in assays for detection or quantitation of analytes. For this purpose, the oligonucleotide can include a detection portion. A detection portion is a region of an oligonucleotide that can be used to mediate detection of the oligonucleotide. Where multiple oligonucleotides are coupled to an antibody fragment, the detection portions of the oligonucleotides can be the same or different. For example, the detection portions can have the same or different nucleotide sequences.

Reactive Chemical Groups

Reactive chemical groups are atoms or moieties that can react with other atoms or moieties to form a covalent bond. Such reactive groups are well known and have generally established chemistries. Preferred reactive chemical groups include sulfhydryl groups, amino groups, carboxyl groups, and imidazole groups. Sulfhydryl groups are preferred reactive chemical groups. The sulfhydryl group can be on a cysteine residue. The reactive chemical group preferably is in the hinge region of the antibody fragment.

The antibody conjugate can be made, for example, by reacting a maleimide-derivatized form of the biomolecule with the antibody fragment. as a result, the biomolecule is coupled to the antibody fragment via the maleimide group coupled to a sulfhydryl group on the antibody fragment. The biomolecule is coupled to the antibody fragment via a maleimide group coupled to a sulfhydryl group in the hinge region of the antibody fragment. The antibody fragment can be made by reducing an antibody, thus producing a reduced or half antibody. The maleimide-derivatized biomolecule can be made by derivatizing an amine biomolecule with maleimide.

Solid Supports

Solid supports are solid-state substrates or supports with which antibody conjugates, analytes or other of the disclosed components can be associated. Antibody conjugates can be associated with solid supports directly or indirectly. For example, antibody conjugates can be directly immobilized on solid supports. It is preferred that antibody conjugates be attached to a solid support via coupling to a reactive chemical group similar to the coupling of biomolecules. The coupling of antibody conjugates to a solid support are preferably resistant to reducing agents. A preferred form of solid support is an array. Another form of solid support is an array detector. An array detector is a solid support to which multiple different antibody conjugates have been coupled in an array, grid, or other organized pattern.

Solid-state substrates for use in solid supports can include any solid material to which antibodies can be coupled. This includes materials such as acrylamide, agarose, latex, cellulose, nitrocellulose, glass, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, and polyamino acids. Solid-state substrates can have any useful form including thin film, membrane, bottles, dishes, slides, fibers, woven fibers, optical fibers, shaped polymers, particles, beads, microparticles, or a combination. Solid-state substrates and solid supports can be porous or non-porous.

Different antibody conjugates can be used together as a set. The set can be used as a mixture of all or subsets of the antibody conjugates used separately in separate reactions, or immobilized in an array. Antibody conjugates used separately or as mixtures can be physically separable through, for example, association with or immobilization on a solid support. An array includes a plurality of antibody conjugates immobilized at identified or predefined locations on the array. Each predefined location on the array generally has one type of antibody conjugate (that is, all the antibody conjugates at that location are the same). Each location will have multiple copies of the antibody conjugate. The spatial separation of different antibody conjugates in the array allows separate detection and identification of analytes.

Although preferred, it is not required that a given array be a single unit or structure. The set of antibody conjugates may be distributed over any number of solid supports. For example, at one extreme, each antibody conjugate may be immobilized in a separate reaction tube or container, or on separate beads or microparticles.

Some solid supports useful in RCA assays have detection antibodies attached to a solid-state substrate. Such antibodies can be specific for a molecule of interest. Captured molecules of interest can then be detected by binding of an antibody conjugate, followed by RCA. Methods for immobilizing antibodies to solid-state substrates are well established and can be used to immobilize the disclosed antibody conjugates and antibody fragments. Immobilization can be accomplished by attachment, for example, to aminated surfaces, carboxylated surfaces or hydroxylated surfaces using standard immobilization chemistries. Examples of attachment agents are cyanogen bromide, succinimide, aldehydes, tosyl chloride, avidin-biotin, photocrosslinkable agents, epoxides and maleimides. A preferred attachment agent is the heterobifunctional cross-linker N-[γ-Maleimidobutyryloxy] succinimide ester (GMBS). These and other attachment agents, as well as methods for their use in attachment, are described in Protein immobilization, fundamentals and applications, Richard F. Taylor, ed. (M. Dekker, New York, 1991), Johnstone and Thorpe, Immunochemistry In Practice (Blackwell Scientific Publications, Oxford, England, 1987) pages 209-216 and 241-242, and Immobilized Affinity Ligands, Craig T. Hermanson et al., eds. (Academic Press, New York, 1992). Antibody conjugates and antibody fragments can be attached to a substrate by chemically cross-linking a free amino group on the antibody to reactive side groups present within the solid-state substrate. For example, antibody conjugates and antibody fragments may be chemically cross-linked to a substrate that contains free amino, carboxyl, or sulfur groups using glutaraldehyde, carbodiimides, or GMBS, respectively, as cross-linker agents. In this method, aqueous solutions containing free antibodies are incubated with the solid-state substrate in the presence of glutaraldehyde or carbodiimide.

A preferred method for attaching antibodies or other proteins to a solid-state substrate is to functionalize the substrate with an amino- or thiol-silane, and then to activate the functionalized substrate with a homobifunctional cross-linker agent such as (Bis-sulfo-succinimidyl suberate (BS³) or a heterobifunctional cross-linker agent such as GMBS. For cross-linking with GMBS, glass substrates are chemically functionalized by immersing in a solution of mercaptopropyltrimethoxysilane (1% vol/vol in 95% ethanol pH 5.5) for 1 hour, rinsing in 95% ethanol and heating at 120° C. for 4 hrs. Thiol-derivatized slides are activated by immersing in a 0.5 mg/ml solution of GMBS in 1% dimethylformamide, 99% ethanol for 1 hour at room temperature. Antibodies or proteins are added directly to the activated substrate, which are then blocked with solutions containing agents such as 2% bovine serum albumin, and air-dried. Other standard immobilization chemistries are known by those of skill in the art.

Each antibody conjugate (or antibody fragment) immobilized on the solid support preferably is located in a different predefined region of the solid support. Each of the different predefined regions can be physically separated from each other of the different regions. The distance between the different predefined regions of the solid support can be either fixed or variable. For example, in an array, each of the components can be arranged at fixed distances from each other, while components associated with beads will not be in a fixed spatial relationship. In particular, the use of multiple solid support units (for example, multiple beads) will result in variable distances.

Components can be associated or immobilized on a solid support at any density. Components preferably are immobilized to the solid support at a density exceeding 400 different components per cubic centimeter. Arrays of components can have any number of components. For example, an array can have at least 1,000 different components immobilized on the solid support, at least 10,000 different components immobilized on the solid support, at least 100,000 different components immobilized on the solid support, or at least 1,000,000 different components immobilized on the solid support.

Antibodies

Antibody fragments for use in the disclosed compositions and methods can be derived from any antibody from any source. For example, useful antibodies include crude (serum) antibodies, purified antibodies, monoclonal antibodies, polyclonal antibodies, recombinant antibodies, and synthetic antibodies. Antibodies specific for antigens and analytes or interest are preferred as a source of antibody fragments.

Antigens have regions called epitopes which make up the specific molecular determinants for antibody:antigen binding. Typically an epitope of a protein is composed of between three or four and eight amino acids (see Watson et al., “Certain Properties Make Substances Antigenic,” in Molecular Biology of the Gene, Fourth Edition, page 836, paragraph 3, (The Benjamin/Cummings Publishing Company, Menlo Park, 1987)). The antigens can contain the entire native epitope, or portions thereof sufficient to react with antibody.

Antibodies can be obtained commercially or produced using well-established methods. For example, Johnstone and Thorpe, on pages 30-85, describe general methods useful for producing both polyclonal and monoclonal antibodies. The entire book describes many general techniques and principles for the use of antibodies in assay systems. An antibody to an antigen of choice can be produced according to Kohler and Milstein, Nature, 256:495-497 (1975), Eur. J. Immunol. 6:511-519 (1976), by immunizing a host with the antigen of choice. Once a host is immunized with the antigen, B-lymphocytes that recognize the antigen are stimulated to grow and produce antibody to the antigen. A collection of the sera containing the antibodies produced by these B-lymphocytes contains the disclosed antibodies that can be used in the disclosed methods.

Polyclonal antibodies can be produced by injecting an animal of choice (such as a rabbit or mouse) with the antigen of choice. The animal is maintained under conditions so that the antibody:antigen complexes are formed. Once these complexes are formed and reach the desired titer, the blood of the animal is collected. The serum containing the polyclonal antibodies (antisera) is separated from the other blood components using any one of a number of procedures, such as affinity separation. The polyclonal antibody-containing serum can optionally be further separated into fractions of particular types of antibodies (e.g., IgG or IgM) or monospecific antibodies can be affinity purified from polyclonal antibody containing serum.

Each activated B-cell, produces clones which in turn produce the monoclonal antibody. B-cells cannot be cultured indefinitely, however, and so a hybridoma must be produced. Hybridomas are produced using the methods developed by Kohler and Milstein, Nature, 256:495-497 (1975).

Hybridomas can be produced by fusing the B-cells obtained by the host organism's spleen to engineered myeloma cells. These cells often have a selectable marker which prevents them from growing in a medium, if they have not been fused to a B-cell. Likewise, B-cells are not immortal and so those that are unfused will die. Thus, the only cells left after fusion are those cells which have come from a successful B-cell and myeloma cell fusion. The fusion cells are analyzed to determine if the desired antibody is being produced by a given fused cell, by for example, testing the fused cells with the antigen in an ELISA assay. The antibodies produced and isolated by this method are specific for a single antigen or epitope on an antigen.

A cell bound enzyme linked immmunosorbent assay (ELISA) can be used to screen supernatants from growing hybridomas (Glassy and Surh, J. Immunol. Method, 81:115 (1985)). Cells which bind the antibody or produce the antibody can be analyzed using Flow Cytometry. Cell surface antigens are detectable by flow cytometry.

While the in vivo use of a monoclonal antibody from a foreign donor species in a different host recipient species is usually uncomplicated, an antigenic site on the donor antibody can cause an adverse immunological response in the organism receiving the donor antibody. The adverse response may serve to hinder the molecular interaction of the donor antibody or acceptance of the donor antibody. There are three preferred ways to produce monoclonal antibodies to be used in humans: humanized mouse antibodies (Winter and Harris, Trends Pharmacol. Sci. 14:139 (1993) and Queen et al. Proc. Natl. Acad. Sci. U.S.A. 86:10029 (1989)), nude mice produced human antibodies (Bruggermann and Neuberger, Immuno. Today 8:391 (1996)), and phage display techniques (Huse et al. Science 246:1275 (1989), Hoogenboom et al. Immunotechnology 4:1 (1998), and Rodi and Makowski, Curr. Opin. Biotechnology 10:87 (1999)). These techniques can be adapted to produce antibody fragments for use in the disclosed antibody conjugates Humanized mouse or chimeric antibodies can be used to reduce or eliminate the adverse host response (Sun et al., Hybridoma, 5 (Supplement 1):S17, 1986; Oi et al., Bio Techniques, 4(3): 214, 1986). Chimeric antibodies are antibodies in which the various domains of the antibodies' heavy and light chains are coded for by DNA from more than one species. Typically, a chimeric antibody will comprise the variable domains of the heavy (VH) and light (VL) chains derived from the donor species producing the antibody of desired antigenic specificity, and the variable domains of the heavy (CH) and light (CL) chains derived from the host recipient species. It is believed that by reducing the exposure of the host immune system to the antigenic determinants of the donor antibody domains, especially those in the CH region, the possibility of an adverse immunological response occurring in the recipient species will be reduced. Thus, for example, it is possible to produce a chimeric antibody for in vivo clinical use in humans which comprises rabbit VH and VL domains coded for by DNA isolated from a rabbit that binds an antigen or an antigen fragment and CH and CL domains coded for with DNA isolated from a human immune system cell. These techniques can be adapted to produce antibody fragments for use in the disclosed antibody conjugates.

Rolling Circle Amplification

Rolling circle amplification (RCA) is a preferred method for amplification of signal from, and detection of, the disclosed antibody conjugates. RCA involves replication of circular single-stranded DNA molecules. In RCA, a rolling circle replication primer hybridizes to amplification target circles followed by rolling circle replication of the amplification target circles using a strand-displacing DNA polymerase. Amplification can take place during rolling circle replication in a single reaction cycle. Rolling circle replication results in large DNA molecules containing tandem repeats of the amplification target circle sequence. This DNA molecule is referred to as a tandem sequence DNA (TS-DNA). Rolling circle amplification is described in detail in U.S. Pat. No. 6,143,495 to Lizardi et al.

A rolling circle replication primer (RCRP) is an oligonucleotide having sequence complementary to the primer complement portion of an amplification target circle. This sequence is referred to as the complementary portion of the RCRP. The complementary portion of a RCRP and the cognate primer complement portion can have any desired sequence so long as they are complementary to each other. In general, the sequence of the RCRP can be chosen such that it is not significantly complementary to any other portion of the amplification target circle. The complementary portion of a rolling circle replication primer can be any length that supports specific and stable hybridization between the primer and the primer complement portion. Generally this is 12 to 100 nucleotides long, but is preferably 20 to 45 nucleotides long.

It is preferred that rolling circle replication primers also contain additional sequence at the 5′ end of the RCRP that is not complementary to any part of the amplification target circle. This sequence is referred to as the non-complementary portion of the RCRP. The non-complementary portion of the RCRP, if present, serves to facilitate strand displacement during DNA replication. The non-complementary portion of a RCRP may be any length, but is generally 1 to 100 nucleotides long, and preferably 4 to 8 nucleotides long. A rolling circle replication primer can be used as the tertiary DNA strand displacement primer in strand displacement cascade amplification.

An amplification target circle (ATC) is a circular single-stranded DNA molecule, generally containing between 40 to 1000 nucleotides, preferably between about 50 to 150 nucleotides, and most preferably between about 50 to 100 nucleotides. Portions of ATCs have specific functions making the ATC useful for rolling circle amplification (RCA). These portions are referred to as the primer complement portion, the detection tag portions, the secondary target sequence portions, the address tag portions, and the promoter portion. The primer complement portion is a required element of an amplification target circle. Detection tag portions, secondary target sequence portions, address tag portions, and promoter portions are optional. Generally, an amplification target circle is a single-stranded, circular DNA molecule comprising a primer complement portion. Those segments of the ATC that do not correspond to a specific portion of the ATC can be arbitrarily chosen sequences. It is preferred that ATCs do not have any sequences that are self-complementary. It is considered that this condition is met if there are no complementary regions greater than six nucleotides long without a mismatch or gap. It is also preferred that ATCs containing a promoter portion do not have any sequences that resemble a transcription terminator, such as a run of eight or more thymidine nucleotides.

An amplification target circle, when replicated, gives rise to a long DNA molecule containing multiple repeats of sequences complementary to the amplification target circle. This long DNA molecule is referred to herein as tandem sequence DNA (TS-DNA). TS-DNA contains sequences complementary to the primer complement portion and, if present on the amplification target circle, the detection tag portions, the secondary target sequence portions, the address tag portions, and the promoter portion. These sequences in the TS-DNA are referred to as primer sequences (which match the sequence of the rolling circle replication primer), spacer sequences (complementary to the spacer region), detection tags, secondary target sequences, address tags, and promoter sequences. Amplification target circles can be used as biomolecules coupled to antibody fragments.

Ligation-mediated rolling circle amplification involves a ligation operation and an amplification operation. The ligation operation circularizes a specially designed nucleic acid probe molecule. This step is dependent on hybridization of the probe to a target sequence and forms circular molecules. The amplification operation is rolling circle replication of the circularized probe. Ligation-mediated rolling circle amplification is described in detail in U.S. Pat. No. 6,143,495 to Lizardi et al. In the disclosed method, the target sequence that mediates circularization of the probe is an nucleic acid coupled to an antibody fragment.

### Methods

The disclosed antibody conjugates can be made generally as described elsewhere herein. The disclosed antibody conjugates can be used for any purpose, and can be put to any use, for which antibodies and antibody compositions can be used. In particular, the disclosed antibody conjugates can be used to associate the coupled biomolecules to any antigen to which the antibody fragment can bind or interact. The disclosed antibody conjugates can be used in analytic methods, including methods for detecting and quantitating, or involving detection or quantitation, of antigens and analytes. The disclosed antibody conjugates can also be used in diagnostic and therapeutic methods. In general, the disclosed antibody conjugates can be used to detect analytes by bringing into contact a antibody conjugate and a sample under conditions that allow interaction of the antibody conjugate and an analyte, where the antibody fragment is specific for the analyte.

Using Antibody Conjugates In Vitro

The disclosed antibody conjugates are suited for use in immunoassays in which they can be utilized in liquid phase or bound to a solid phase carrier. Such assays are enhanced by the presence of biomolecules that can be used for separation, capture and/or detection. Examples of types of immunoassays which can utilize the disclosed antibody conjugates are competitive and noncompetitive immunoassays in either a direct or indirect format. Detection of analyte using the disclosed antibody conjugates can be done utilizing immunoassays which are run in either the forward, reverse, or simultaneous modes, including immunohistochemical assays on physiological samples. Preferred assays involve association of antibody conjugates with antigens or analytes, followed by detection of biomolecules present in the antibody conjugates.

As used in this invention, the term “epitope” is meant to include any determinant capable of specific interaction with the antibody fragments in the disclosed antibody conjugates. Epitopic determinants usually consist of chemically active surface groupings of molecules such as amino acids or sugar side chains and usually have specific three dimensional structural characteristics, as well as specific charge characteristics.

Detecting Analytes

Analytes can be detected using the disclosed conjugated antibodies by bringing into contact an antibody conjugate (where the antibody fragment is specific for an analyte) and a sample under conditions that allow interaction of the antibody conjugate and the analyte. The analyte is detected indirectly by detecting the antibody conjugate following contact with the sample. In turn, detection of the antibody conjugate can be mediated by detection of the biomolecule. The biomolecule can be selected for the ease and specificity of its detection.

Oligonucleotides are preferred biomolecules for such detection. The oligonucleotide can be detected by any suitable technique. Many techniques for detecting nucleic acids are known and can be used to detect the disclosed antibody conjugates. The oligonucleotides can be amplified (or can mediate nucleic acid amplification) and then the amplification product can be detected. A preferred form of amplification is rolling circle amplification. For rolling circle amplification, it is preferred that the oligonucleotide be a rolling circle replication primer, an amplification target circle, or a target sequence.

Using Antibody Conjugates In Vivo

The disclosed antibody conjugates can be used in vivo. For example, the antibody conjugates can be used for therapeutic or diagnostic purposes. For this purpose, drugs or cytotoxic agents can be used as the biomolecule. Examples of therapeutic agents which can be coupled to the disclosed antibody conjugates are drugs, radioisotopes, lectins, and toxins or agents which will covalently attach the antibody conjugate to the mema.

Toxins are poisonous substances produced by plants, animals, or microorganisms that, in sufficient dose, are often lethal. Diphtheria toxin is a substance produced by Corynebacterium diphtheria which can be used therapeutically. This toxin consists of an alpha and beta subunit which under proper conditions can be separated. Lectins are proteins, usually isolated from plant material, which bind to specific sugar moieties. Many lectins are also able to agglutinate cells and stimulate lymphocytes. However, ricin is a toxic lectin which can be used immunotherapeutically. This is accomplished by binding the alpha-peptide chain of ricin, which is responsible for toxicity, to the antibody fragment to enable site specific delivery of the toxic effect. Other therapeutic agents which can be coupled to the disclosed antibody fragments are known, or can be easily ascertained, by those of skill in the art.

A mixed toxin molecule is a molecule derived from two different polypeptide toxins. Generally, as discussed above in connection with diphtheria toxin, polypeptide toxins have, in addition to the domain responsible for generalized eukaryotic cell binding, an enzymatically active domain and a translocation domain. The binding and translocation domains are required for cell recognition and toxin entry respectively. Naturally-occurring proteins which are known to have a translocation domain include diphtheria toxin, Pseudomonas exotoxin A, and possibly other peptide toxins. The translocation domains of diphtheria toxin and Pseudomonas exotoxin A are well characterized (see, e.g., Hoch et al., Proc. Natl. Acad. Sci. USA 82:1692, 1985; Colombatti et al., J. Biol. Chem. 261:3030, 1986; and Deleers et al., FEBS Lett. 160:82, 1983), and the existence and location of such a domain in other molecules may be determined by methods such as those employed by Hwang et al. (Cell 48:129, 1987); and Gray et al. (Proc. Natl. Acad. Sci. USA 81:2645, 1984).

A useful mixed toxin hybrid molecule can be formed by fusing the enzymatically active A subunit of E. coli Shiga-like toxin (Calderwood et al., Proc. Natl. Acad. Sci. USA 84:4364, 1987) to the translocation domain (amino acid residues 202 through 460) of diphtheria toxin, and to a molecule targeting a particular cell type, as described in U.S. Pat. No. 5,906,820 to Bacha. The targeting portion of the three-part hybrid causes the molecule to attach specifically to the targeted cells, and the diphtheria toxin translocation portion acts to insert the enzymatically active A subunit of the Shiga-like toxin into the targeted cell. The enzymatically active portion of Shiga-like toxin, like diphtheria toxin, acts on the protein synthesis machinery of the cell to prevent protein synthesis, thus killing the cell.

In using the disclosed antibody conjugates for the in vivo detection of antigen, the antibody conjugate is given in a dose which is diagnostically effective. The term “diagnostically effective” means that the amount of antibody conjugate is administered in sufficient quantity to enable detection of the antibody conjugate at a site antigen concentration. The concentration of antibody conjugate which is administered should be sufficient such that the binding is detectable compared to the background signal. As a rule, the dosage of antibody conjugate for in vivo diagnosis will vary depending on such factors as age, sex and extent of disease of the individual. The dosage of antibody conjugate can vary from about 0.01 mg/m² to about 20 mg/m², preferably about 0.1 mg/m² to about 10 mg/m².

The disclosed antibody conjugates can be used to monitor the course of treatment in an individual. Thus, by measuring the increase or decrease in the amount or concentration of an antigen associated with a disease or condition, it would be possible to determine whether a particular therapeutic regimen aimed at ameliorating the immune response mediated disorder is effective.

The term “ameliorate” denotes a lessening of the detrimental affect of a condition or disorder in the animal receiving therapy. The term “therapeutically effective” means that the amount of antibody conjugate used is of sufficient quantity to ameliorate the cause of disease. The drugs with which can be conjugated to the antibody fragments include compounds which are classically referred to as drugs such as for example, mitomycin C, daunorubicin, and vinblastine. Other therapeutic agents which can be coupled to antibody fragments are known, or can be easily ascertained, by those of skill in the art.

The dosage ranges for the administration of the disclosed antibody conjugates are those large enough to produce the desired effect in which the symptoms of the condition or disorder are ameliorated. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary from about 0.1 mg/m² to about 2000 mg/m², preferably about 0.1 mg/M² to about 500 mg/M²/dose, in one or more dose administrations daily, for one or several days. Generally, when the antibody conjugates are administered lower dosages, as compared those used for in vivo immunodiagnostic imaging, can be used.

The disclosed antibody conjugates can be administered parenterally by injection or by gradual perfusion over time. The disclosed antibody conjugates can be administered intravenously, intraperitoneally, intramuscularly, subcutaneously, intracavity, or transdermally. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

### EXAMPLES

The present invention will now be further described by way of the following non-limiting examples. In applying the disclosure of these examples, it should be kept clearly in mind that other and different embodiments of the methods disclosed according to the present invention will no doubt suggest themselves to those of skill in the relevant art.

### Example 1

### Preparation of a Half Antibody DNA Conjugate

Mouse anti-biotin monoclonal IgG (Jackson ImmunoResearch Laboratories, Inc.) at a concentration of 5 mg/ml in 1×PBS buffer (pH 7.2) with 10 mM EDTA was reduced with 50 mM 2-mercaptoethylamine (MEA, Pierce Chemical Co.) to cleave the difulfide bonds in the hinge region of the IgG structure and provide free sulfliydryl groups by incubation for 90 min at 37° C. The reduced IgG was purified by PDIO column (Amersham Pharmacia Biotech) using 1×PBS buffer (pH 7.2) with 10 mM EDTA at 4° C. to remove free MEA. Fractions containing antibody were determined by BCA assay, pooled and then concentrated using Centricon YM-30.

5′-terminal amine-modified oligo (primer 1) was synthesized on an automated DNA synthesizer and treated with 10 fold molar excess of N-[γ-maleimidobutyryloxy]sulfo-succinimide ester (sulfo-GMBS, Pierce Chemical Co.) in 1×PBS buffer (pH 7.2). The reaction was incubated for 30 mm at 37° C. and then 30 min at room temperature. The maleimide-activated oligo was purified by a PD10 column to remove excess GMBS. Fractions containing modified oligo were determined by UV absorbance at 260 nm, and collected. The pool of activated oligo was concentrated by using a Centricon YM-3 spin column.

The derivatized oligo was then conjugated to the reduced IgG (molar ratio of modified oligo to reduced IgG was 10: 1) by incubation for 2 hrs at room temperature with shaking. The conjugate was then purified by superdex 200 gel filteration column (Amersham Pharmacia Biotech). The purity of conjugate was determined by agarose gel and SDS page.

### Example 2

### Preparation of a Second Generation Half Antibody DNA Conjugate by Thiolation Followed by Reduction and DNA Conjugation.

A mouse anti-biotin monoclonal IgG (Jackson ImmunoResearch Laboratories, Inc.) at a concentration of 5 mg/ml in 1×PBS buffer (pH 7.2) with 10 mM EDTA was thiolated with a 20 fold molar excess of 2-imminothiolane-HCl (Traut's reagent, Pierce Chemical Co.) in 1×PBS (pH 7.2)/10 mM EDTA at room temperature for 1 hr. Iminothiolated IgG was separated from excess Traut's reagents by a PD-10 column equilibrated with 1×PBS (pH 7.2)/10 mM EDTA buffer at 4° C. Fractions containing thiolated IgG were determined by BCA assay, then collected, and concentrated.

Thiolated IgG was reduced with 50 mM 2-mercaptoethylamine (MEA, Pierce Chemical Co.) to cleave the difulfide bonds in the hinge region of the IgG structure and provide additional free sulfhydryl groups by incubation for 90 min at 37° C. The reduced IgG was purified by PD-10 column (Amersham Pharmacia Biotech) using 1×PBS buffer (pH 7.2) with 10 mM EDTA at 4° C. to remove free MEA. Fractions containing reduced antibody were determined by BCA assay, pooled and then concentrated using Centricon YM-30.

5′-terminal amine-modified oligo (primer 1) was synthesized in house and treated with 10 fold molar excess of N-[y-maleimidobutyryloxy]sulfo-succinimide ester (sulfo-GMBS, Pierce Chemical Co.) in 1×PBS buffer (pH 7.2). The reaction was incubated for 30 min at 37° C. and then 30 min at room temperature. The maleimide-activated oligo was purified by the PD-10 column to remove excess GMBS. Fractions containing modified oligo were determined by UV absorbance at 260 nm, and collected. The pool of activated oligo was concentrated by using Centricon YM-3.

The derivatized oligo was then conjugated to the reduced thiolated IgG (molar ratio of modified oligo to reduced thiolated IgG was 10:1) by incubation for 2 hrs at room temperature with shaking. The conjugate was then purified by superdex 200 gel filteration column (Amersham Pharmacia Biotech). The purity of conjugate was determined by agarose gel and SDS page.

### Example 3

### Comparison of Antibody Fragment Conjugates with Whole Antibody Conjugates for Detection of Analytes on Microarrays

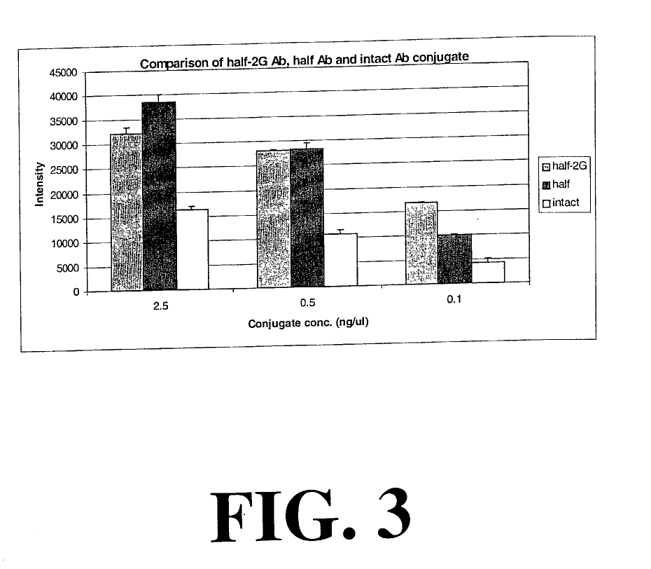

Glass slides were functionalized with thiol-silane and activated with GMBS. Serial dilution of cy5-BSA (0.2 mg/ml, 0.1 mg/ml and 0.5 mg/ml) and biotinylated BSA (2 ug/ml, 200 ng/ml, and 20 ng/ml) were spotted onto the slides by using a pin-tool type microarrayer. Each microarray was blocked by adding 30 ul of a 2 mg/ml BSA solution in 50 mM glycine (pH 9.0) and incubating for 1 hr at 37° C. in a humidity chamber. After blocking, slides were twice washed in 1×PBS/0.05% Tween 20 for two minutes. Mouse monoclonal anti-biotin half Ab second generation conjugate, half Ab conjugate and intact Ab conjugate of primer 1 were diluted to 2.5 ug/ml, 0.5 ug/ml and 0.1 ug/ml, and were preannealed with 50 nM circle 1 in 1×PBS/0.05% Tween20/2 mM EDTA at 37° C. for 30 min. 20 ul was applied to each specific array and incubated at 37° C. for 30 min in a humid chamber, and then slides were washed twice. 20 ul of RCA reaction solution containing T7 native DNA polymerase (0.01 units/ul)/1 mM dNTPs/0.03 mg/ml single-stranded DNA-binding protein/1×sequenase/8% DMSO/0.05 uM DNA decorator was then added to each microarray. The slides were incubated at 37° C. for 45 min. At the same time, decorator was hybridized to the RCA product. Slides were washed once in 1×PBS/0.05% Tween20, and once in 1×PBS/0.05% Tween 20, and then spin-dried. Slides were scanned on GSI ScanArray Lite microarray scanner, and fluorescence was quantitated using Quantarray software. The results are shown in FIG. 3.

It is understood that the disclosed invention is not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, an and “the” include plural reference unless the context clearly dictates otherwise. Thus, for example, reference to “a host cell” includes a plurality of such host cells, reference to “the antibody” is a reference to one or more antibodies and equivalents thereof known to those skilled in the art, and so forth.

Unless defined otherwise, all technical and scientific terms used herein have the same meanings as commonly understood by one of skill in the art to which the disclosed invention belongs. Although any methods and materials similar or equivalent to those described herein can be used in the practice or testing of the present invention, the preferred methods, devices, and materials are as described. Publications cited herein and the material for which they are cited are specifically incorporated by reference. Nothing herein is to be construed as an admission that the invention is not entitled to antedate such disclosure by virtue of prior invention.

Those skilled in the art will recognize, or be able to ascertain using no more than routine experimentation, many equivalents to the specific embodiments of the invention described herein. Such equivalents are intended to be encompassed by the following claims.

I claim:

1. A composition comprising an antibody fragment and a biomolecule,
wherein the biomolecule is coupled to the antibody fragment via a reactive chemical group, wherein the coupling between the biomolecule and the antibody fragment is resistant to reducing agents.

2. The composition of claim 1 wherein the reactive chemical group is a sulfhydryl group, an amino group, a carboxyl group, or an imidazole group.

3. The composition of claim 2 wherein the reactive chemical group is a sulfhydryl group.

4. The composition of claim 3 wherein the sulfhydryl group is on a cysteine residue.

5. The composition of claim 1 wherein the reactive chemical group is in the hinge region of the antibody fragment.

6. The composition of claim 1 wherein the antibody fragment is a half antibody or a F(ab′)₂.

7. The composition of claim 6 wherein the half antibody is produced by reducing an antibody to break disulfide bonds.

8. The composition of claim 1 wherein the biomolecule is a nucleic acid, a protein, a carbohydrate, an oligonucleotide, an oligopeptide, an oligosaccharide, a peptide, a hapten, or an aptamer.

9. The composition of claim 8 wherein the biomolecule is a nucleic acid.

10. The composition of claim 1 further comprising a second biomolecule,
wherein the second biomolecule is coupled to the antibody fragment via a second reactive chemical group, wherein the coupling between the second biomolecule and the antibody fragment is resistant to reducing agents.

11. The composition of claim 10 wherein the second reactive chemical group is a sulfhydryl group, an amino group, a carboxyl group, or an imidazole group.

12. The composition of claim 11 wherein the second reactive group is a sulfhydryl group.

13. The composition of claim 12 wherein the sulfhydryl group is on a cysteine residue.

14. The composition of claim 10 wherein the second reactive chemical group is in the hinge region of the antibody fragment.

15. The composition of claim 10 wherein the first biomolecule and the second biomolecule have the same structure.

16. The composition of claim 10 wherein the first biomolecule and the second biomolecule have different structures.

17. The composition of claim 1 wherein the antibody fragment is specific for an analyte.

18. The composition of claim 17 wherein the analyte is a protein or peptide.

19. The composition of claim 18 wherein the protein or peptide is a protein or peptide associated with a disease or condition.

20. The composition of claim 1 wherein the biomolecule is coupled to the antibody fragment via a maleimide group coupled to a sulfhydryl group in the hinge region of the antibody fragment.

21. The composition of claim 1 wherein the composition is made by reacting a maleimide-derivatized form of the biomolecule with the antibody fragment,
wherein the biomolecule is coupled to the antibody fragment via the maleimide group coupled to a sulfhydryl group in the hinge region of the antibody fragment.

22. The composition of claim 1 wherein the biomolecule is an oligonucleotide.

23. The composition of claim 22 further comprising a second oligonucleotide,
wherein the second oligonucleotide is coupled to the antibody fragment via a second reactive chemical group, wherein the coupling between the second oligonucleotide and the antibody fragment is resistant to reducing agents.

24. The composition of claim 23 wherein the first oligonucleotide and the second oligonucleotide each comprise a detection portion, wherein the detection portions of the first oligonucleotide and the second oligonucleotide have different nucleotide sequences.

25. The composition of claim 23 wherein the first oligonucleotide and the second oligonucleotide have the same nucleotide sequence.

26. The composition of claim 23 wherein the first oligonucleotide and the second oligonucleotide have different nucleotide sequences.

27. The composition of claim 22 further comprising tandem sequence DNA,
wherein the tandem sequence DNA is coupled to the oligonucleotide.

28. The composition of claim 27 wherein the tandem sequence DNA is produced by rolling circle replication of an amplification target circle, wherein the oligonucleotide primes the rolling circle replication.

29. The composition of claim 22 wherein the oligonucleotide comprises a primer.

30. The composition of claim 29 wherein the primer is a rolling circle replication primer.

31. The composition of claim 22 wherein the oligonucleotide comprises an amplification target circle.

32. A composition comprising an antibody fragment and a biomolecule,
wherein the biomolecule is coupled to the antibody fragment via a maleimide group coupled to a reactive chemical group, wherein the coupling between the biomolecule and the antibody fragment is resistant to reducing agents.

33. The composition of claim 32 wherein the biomolecule is an oligonucleotide.

34. The composition of claim 33 further comprising a second oligonucleotide,
wherein the second oligonucleotide is coupled to the antibody fragment via a second reactive chemical group, wherein the coupling between the second oligonucleotide and the antibody fragment is resistant to reducing agents.

35. A composition comprising an antibody fragment and a biomolecule, wherein the composition is made by
reacting a maleimide-derivatized biomolecule with an antibody fragment,
wherein the biomolecule is coupled to the antibody fragment via the maleimide group coupled to a reactive chemical group, wherein the coupling between the biomolecule and the antibody fragment is resistant to reducing agents.

36. The composition of claim 35 wherein the biomolecule is an oligonucleotide.

37. A method of making an antibody conjugate, the method comprising
reacting a maleimide-derivatized biomolecule with an antibody fragment,
wherein the biomolecule is coupled to the antibody fragment via the maleimide group coupled to a reactive chemical group, wherein the coupling between the biomolecule and the antibody fragment is resistant to reducing agents.

38. The method of claim 37 further comprising
reducing an antibody to produce the antibody fragment.

39. The method of claim 37 further comprising
derivatizing an amine biomolecule with maleimide to produce the maleimide-derivatized biomolecule.

40. The method of claim 39 further comprising
producing the amine biomolecule.

41. The method of claim 37 further comprising
reducing an antibody to produce the antibody fragment.

42. The method of claim 37 wherein the biomolecule is an oligonucleotide.

43. A method of detecting analytes, the method comprising
bringing into contact a antibody conjugate and a sample under conditions that allow interaction of the antibody conjugate and an analyte,
wherein the antibody conjugate comprises an antibody fragment and a biomolecule,
wherein the biomolecule is coupled to the antibody fragment via a reactive chemical group, wherein the coupling between the biomolecule and the antibody fragment is resistant to reducing agents,
wherein the antibody fragment is specific for the analyte.

44. The method of claim 43 wherein the reactive chemical group is a sulhydryl group, an amino group, a carboxyl group, or an imidazole group.

45. The method of claim 44 wherein the reactive chemical group is a sulfhydryl group.

46. The method of claim 45 wherein the sulfhydryl group is on a cysteine residue.

47. The method of claim 43 wherein the reactive chemical group is in the hinge region of the antibody fragment.

48. The method of claim 43 wherein the antibody fragment is a half antibody or a F(ab′)₂.

49. The method of claim 48 wherein the half antibody is produced by reducing an antibody to break disulfide bonds.

50. The method of claim 43 wherein the biomolecule is a nucleic acid, a protein, a carbohydrate, an oligonucleotide, an oligopeptide, an oligosaccharide, a peptide, a hapten, or an aptamer.

51. The method of claim 50 wherein the biomolecule is a nucleic acid.

52. The method of claim 43 wherein the antibody conjugate further comprises a second biomolecule,
wherein the second biomolecule is coupled to the antibody fragment via a second reactive chemical group, wherein the coupling between the second biomolecule and the antibody fragment is resistant to reducing agents.

53. The method of claim 52 wherein the second reactive chemical group is a sulfhydryl group, an amino group, a carboxyl group, or an imidazole group.

54. The method of claim 53 wherein the second reactive group is a sulfhydryl group.

55. The method of claim 54 wherein the sulfhydryl group is on a cysteine residue.

56. The method of claim 52 wherein the second reactive chemical group is in the hinge region of the antibody fragment.

57. The method of claim 52 wherein the first biomolecule and the second biomolecule have the same structure.

58. The method of claim 52 wherein the first biomolecule and the second biomolecule have different structures.

59. The method of claim 43 wherein the antibody fragment is specific for the analyte.

60. The method of claim 59 wherein the analyte is a protein or peptide.

61. The method of claim 60 wherein the protein or peptide is a protein or peptide associated with a disease or condition.

62. The method of claim 43 further comprising
reducing an antibody to produce the antibody fragment.

63. The method of claim 43 further comprising
derivatizing an amine biomolecule with maleimide to produce the maleimide-derivatized biomolecule.

64. The method of claim 63 further comprising
producing the amine biomolecule.

65. The method of claim 43 further comprising
reducing an antibody to produce the antibody fragment.

66. The method of claim 43 wherein the biomolecule is an oligonucleotide.

67. The method of claim 66 wherein the antibody conjugate further comprises a second oligonucleotide,
wherein the second oligonucleotide is coupled to the antibody fragment via a second reactive chemical group, wherein the coupling between the second oligonucleotide and the antibody fragment is resistant to reducing agents.

68. The method of claim 67 wherein the first oligonucleotide and the second oligonucleotide each comprise a detection portion, wherein the detection portions of the first oligonucleotide and the second oligonucleotide have different nucleotide sequences.

69. The method of claim 67 wherein the first oligonucleotide and the second oligonucleotide have the same nucleotide sequence.

70. The method of claim 67 wherein the first oligonucleotide and the second oligonucleotide have different nucleotide sequences.

71. The method of claim 66 wherein the antibody conjugate further comprises tandem sequence DNA,
wherein the tandem sequence DNA is coupled to the oligonucleotide.

72. The method of claim 71 wherein the tandem sequence DNA is produced by rolling circle replication of an amplification target circle, wherein the oligonucleotide primes the rolling circle replication.

73. The method of claim 66 wherein the oligonucleotide comprises a primer.

74. The method of claim 73 wherein the primer is a rolling circle replication primer.

75. The method of claim 71 further comprising rolling circle replication of an amplification target circle to produce tandem sequence DNA, wherein the oligonucleotide mediates rolling circle replication of the amplification target circle.

76. The method of claim 75 wherein the oligonucleotide is a rolling circle replication primer that primes the rolling circle replication.

77. The method of claim 66 wherein the oligonucleotide comprises an amplification target circle.

78. A set of antibody conjugates, wherein each antibody conjugate comprises an antibody fragment and a biomolecule,
wherein the biomolecule is coupled to the antibody fragment via a reactive chemical group, wherein the coupling between the biomolecule and the antibody fragment is resistant to reducing agents.

79. The set of claim 78 wherein each biomolecule of each antibody conjugate is different.

80. The set of claim 78 wherein the set comprises a plurality of different antibody conjugates.

81. The set of claim 78 wherein the biomolecule of at least one of the antibody conjugates is an oligonucleotide.

82. A composition comprising a antibody conjugate and a solid support, wherein the antibody conjugate comprises an antibody fragment and a biomolecule,
wherein the biomolecule is coupled to the antibody fragment via a reactive chemical group, wherein the coupling between the biomolecule and the antibody fragment is resistant to reducing agents.

83. The composition of claim 82 wherein the compoition comprises a plurality of antibody conjugates.

84. The composition of claim 82 wherein the biomolecule is an oligonucleotide.

85. The composition of claim 82 wherein the composition comprises a plurality of antibody conjugates, wherein each of the antibody conjugates is located in a different predefined region of the solid support.

86. The composition of claim 85 wherein the distance between the different predefined regions of the solid support is fixed.

87. The composition of claim 86 wherein the solid support comprises thin film, membrane, bottles, dishes, slides, fibers, woven fibers, optical fibers, shaped polymers, particles, beads, microparticles, or a combination.

88. The composition of claim 85 wherein the distance between at least two of the different predefined regions of the solid support is variable.

89. The composition of claim 88 wherein the solid support comprises at least one thin film, membrane, bottle, dish, slide, fiber, woven fiber, optical fiber, shaped polymer, particle, bead, or microparticle.

90. The composition of claim 89 wherein the solid support comprises at least two thin films, membranes, bottles, dishes, slides, fibers, woven fibers, optical fibers, shaped polymers, particles, beads, microparticles, or a combination.

91. The composition of claim 85 wherein the antibody conjugates collectively correspond to a plurality of analytes.

92. The composition of claim 82 wherein the solid support comprises thin film, membrane, bottles, dishes, slides, fibers, woven fibers, optical fibers, shaped polymers, particles, beads, microparticles, or a combination.

93. The composition of claim 82 wherein the solid support comprises acrylamide, agarose, latex, cellulose, nitrocellulose, glass, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, or polyamino acids.

94. The composition of claim 82 wherein the solid support is porous.
